(19) United States
(12) Patent Application Publication
(10) Pub. No.: US 2025/0312021 A1
(43) Pub. Date: Oct. 9, 2025

(54) MINIMALLY INVASIVE LEFT ATRIAL APPENDAGE CLIP

(71) Applicant: Syntheon 2.0, LLC, Miami, FL (US)

(72) Inventors: Derek Dee DEVILLE, Coral Gables, FL (US); William Thomas Bales, Miami, FL (US); Richard Cartledge, Boca Raton, FL (US); Alejandro N. Calderon, Miami, FL (US); Michael Walter Kirk, Miami, FL (US); Michael Sean McBrayer, Coral Gables, FL (US); Matthew Palmer, Coral Gables, FL (US); Carlos Rivera, Cooper City, FL (US); Vincent Turturro, Coral Gables, FL (US)

(21) Appl. No.: 19/170,452

(22) Filed: Apr. 4, 2025

Related U.S. Application Data
(60) Provisional application No. 63/575,247, filed on Apr. 5, 2024.

(51) Int. Cl. A61B 17/00

(52) U.S. Cl. CPC A61B 17/0057; A61B 2017/00367; A61B 2017/00477; A61B 2017/00539; A61B 2017/00557; A61B 2017/00584; A61B 2017/00623; A61B 2017/00862

(57) ABSTRACT

An exclusion clip delivery and recapture system, including an exclusion clip with first and second struts having first and second connector interfaces, and a delivery and recapture device for the exclusion clip. The device includes a handle having a jaw control, a shaft, and an end effector connected to the shaft, including first and second jaws operatively connected to the jaw control to move the jaws with respect to the other, and first and second connectors on the first and second jaws for engagement with the first and second connector interfaces of the exclusion clip. The system may further include a guide mechanism for guiding at least one of the first or second connectors into engagement with the exclusion clip, and/or an actuator for moving at least one of the first or second jaws such as a pulley actuator, a hydraulic actuator, or a screw and nut actuator.

### FIELD OF THE INVENTION

The present disclosure relates to devices, systems, and methods for clipping the left atrial appendage (“LAA”) of the heart to exclude the LAA from the left atrium of the heart, and more particularly to minimally invasive LAA clipping devices, delivery and recapture systems, and methods.

### BACKGROUND

A common type of cardiac arrhythmia is atrial fibrillation (AF), which is characterized as the chaotic and rapid electrical activity of the upper chambers of the heart. There are several causes and risk factors leading to the development of atrial fibrillation, including hypertension, acute and chronic rheumatic heart disease, and hyperthyroidism. Because of this abnormal heart rhythm, contraction of the atrial fibers is asynchronous (not in harmony or unison), such that atrial pumping could cease altogether. Therefore, one of the most dangerous conditions occurring during atrial fibrillation is the disruption or stasis of the blood flow in the atria, which can lead to thrombus (blood clot) formation, placing the afflicted person at a high risk of a heart attack or an embolic stroke.

A majority of blood clots resulting from atrial fibrillation originate in the LAA, due to the LAA's anatomical position and physiological characteristics. The LAA is a pedunculated and finger-shaped, sack-like cavity connected to the lateral wall of the left atrium between the mitral valve and the root of the left pulmonary vein. Thus, the LAA is a prime location for the detrimental pooling and accumulation of stagnant blood when the heart is not contracting to squeeze blood into the ventricles at a normal and coordinated pace. As a result, clots can easily form and accumulate in the LAA, build upon themselves, and propagate out from the LAA and into the atrium. Accordingly, because the LAA is predisposed for thrombus formation, the containment or elimination of clots formed in the LAA of atrial fibrillation patients would greatly reduce the incidence of stroke in those patients.

Pharmacological therapies, such as blood thinners, anticoagulants, and antiplatelet medications are well-known and routinely used to reduce the risk of blood clot formation. However, these medications are oftentimes associated with harmful and distressing side effects and complications, including excessive bleeding, headaches, dizziness, fatigue, and contraindications, making patient compliance and tolerance very difficult.

Another approach to reducing or eliminating the risk of clot formation in the LAA is through a surgical procedure to shut off or substantially restrict blood flow between the LAA and left atrium. One surgical approach is the application of an exclusion clip to the exterior surface of the LAA. An exclusion clip is positioned about and around the base of the LAA to apply a sufficient pinching or clamping pressure that effectively closes the interior fluid passageway between the LAA and the atrium, without ever penetrating the heart. Therefore, the potential for uncontrolled bleeding or other trauma occurring to the heart is reduced as compared to other surgical procedures. Also, because no element of the exclusion clip is introduced into the cardiovascular system, there is minimal risk of inadvertently creating a site that promotes formation of clots in the future.

Exclusion clips employed to isolate the LAA are generally formed from a pair of elongated and opposing clamping members urged together by one or more spring members. Prior to application of the exclusion clip to the LAA, a delivery device engages the exclusion clip and imparts a force counteracting the spring-biased closing force of the spring member or members in order to separate the clamping members from each other and create an interior space therebetween. During application, the LAA is positioned within the interior space of the exclusion clip to be received between the opposing clamping members. Once the surgeon determines that the exclusion clip is in a desirable position with respect to the LAA, the clip's delivery device relieves the counteracting force imparted to the spring member or members and then disengages from the exclusion clip. As a result, the clamping members return to their inwardly spring-biased state to snugly surround the LAA in a grip-like manner and produce a clamping action against the exterior surface of the LAA.

To ensure the effectiveness and safety of the exclusion clip approach to isolating the LAA, the exclusion clip must be positioned accurately with respect to the LAA and the remainder of the heart, and with sufficient pressure, to close off the blood flow into and out from the LAA, while at the same time not severing or otherwise damaging the LAA or any other surrounding structure. Therefore, the surgeon must control the placement of the exclusion clip and determine that the clip is sufficiently closed and securely in place. Once the exclusion clip seats on the LAA, the interposed tissue will desiccate and otherwise shrink and change, thereby requiring a different and greater amount of clamping force to keep the LAA sealed properly.

Improvements have been made to allow for repositioning or removal of the LAA exclusion clip. For example, U.S. Pat. No. 10,925,615, the contents of which are incorporated herein by reference, describes a recapturable LAA clipping device. During the surgical application of the LAA exclusion clip, the LAA exclusion clip may be placed on the LAA and, if the position is not optimal, the LAA exclusion clip may be recaptured by the insertion device and repositioned or removed.

When an exclusion clip is released from the insertion device, its position and/or orientation may change, or the exclusion clip can become at least partially obscured by tissue. This can make it challenging to align the insertion device with the exclusion clip when recapture is necessary. Further, while the insertion of LAA exclusion clips is often done during open-heart surgery, there is a desire to deliver LAA exclusion clips with minimally invasive surgery. Minimizing difficulties in the recapture process is even more important for minimally invasive surgery since complications can result in having to open the patient. Also, for minimally invasive surgery, the end effector and actuation mechanism for opening and closing the exclusion clip may need to be operable at a greater distance from the handle.

Thus, a need exists for improvements over the existing systems, devices, and processes as discussed above.

### SUMMARY

An object of the present invention is to provide improved LAA clipping devices, delivery and recapture systems, and methods.

In one exemplary embodiment according to the present disclosure, an exclusion clip delivery and recapture system is provided, including an exclusion clip including a first strut having a first connector interface and a second strut having a second connector interface and a delivery and recapture device for the exclusion clip. The delivery and recapture device includes a handle including a jaw control, a shaft having a proximal end connected to the handle and an end effector, connected to a distal end of the shaft, including first and second jaws operatively connected to the jaw control to move at least one of the first or second jaws with respect to the other, a first connector on the first jaw for engagement with the first connector interface of the exclusion clip, and a second connector on the second jaw for engagement with the second connector interface of the exclusion clip. A guide mechanism may be provided for guiding at least one of the first or second connectors into engagement with the exclusion clip (e.g., during a recapture of the exclusion clip).

The guide mechanism may include a tether extending from at least one of the first connector or the second connector. In some embodiments, the guide mechanism includes a tether extending from the first connector, through the exclusion clip, to the second connector. The tether may be selectively releasable to disconnect the exclusion clip from the first and second connectors.

In some embodiments, each of the first and second connector interfaces define a cavity having a proximal end with an opening for receiving the respective one of the first or second connector and a closed distal end. The guide mechanism may include a first tether extending from the first connector, a distal end of the first tether being removably attached to the closed distal end of the cavity of the first connector, and/or a second tether extending from the second connector, a distal end of the second tether being removably attached to the closed distal end of the cavity of the second connector.

The distal ends of the first and second tethers include a lock configured to engage in a retention feature in the distal ends of the cavities. In some embodiments, the lock is one of a bendable end piece, a coil, a balloon, a collapsable strut, or a retention clip. In some embodiments, the lock engages in a respective cavity by friction between the lock and the cavity wall without requiring a retention feature in the cavity. In some embodiments, at least one of the first and second connectors has a lock in the form of a distal end that is displaceable laterally in response to tension from a tether.

The guide mechanism may include at least one cone on a proximal end of the exclusion clip or at least one magnet. The magnet may be positioned at the closed distal end of the cavity of at least one of the first or second connector interfaces, and/or at the proximal end of the cavity of at least one of the first or second connector interfaces.

In some embodiments, the guide mechanism includes a flexible extension extending from at least one of the first or second connectors. The flexible extensions may be selectively turnable or bendable in response to a user input.

In some embodiments, the end effector is removably connected to the distal end of the shaft. For example, the distal end of the shaft may include a mechanism to grasp a proximal end of the end effector and release the end effector in response to a user input. In some embodiments, the end effector is rotatably connected to the distal end of the shaft and/or the end effector is telescoping distal to the rotatable connection.

Further provided is a delivery and recapture device for a left atrial appendage (LAA) exclusion clip including a handle, a shaft having a proximal end connected to the handle, wherein the shaft is bendable or turnable in response to a user input, and an end effector connected to a distal end of the shaft. The end effector has first and second jaws operatively configured to move with respect to the other, a first connector extending from a distal end of the first jaw for engagement with a first strut of a LAA exclusion clip, and a second connector extending from a distal end of the second jaw for engagement with a second strut of the LAA exclusion clip. The end effector is releasably connected to a distal end of the shaft. An actuator is provided for moving the at least one of the first or second jaws with respect to the other in response to a user input.

Further provided is a delivery and recapture device for a left atrial appendage (LAA) exclusion clip, including a handle including, a shaft having a proximal end connected to the handle, and an end effector connected to a distal end of the shaft, including first and second jaws operatively connected to a jaw control to move at least one of the first or second jaws with respect to the other, a first connector extending from a distal end of the first jaw for engagement with a first strut of a LAA exclusion clip, and a second connector extending from a distal end of the second jaw for engagement with a second strut of the LAA exclusion clip. An actuator is provided for moving the at least one of the first or second jaws with respect to the other in response to the jaw control, the actuator including one of a pulley actuator, a hydraulic actuator, or a screw and nut actuator. The jaw control may be, for example, a mechanical or electro-mechanical input device on the handle.

In some embodiments, the actuator is the pulley actuator including pulley on an axle, a wire extending over the pulley, a first arm pivotably attached to the first jaw and the axle, and a second arm pivotably attached to the second jaw and the axle, wherein tensioning the wire over the pulley displaces the axle and extends the first and second arms thereby moving the at least one of the first or second jaws with respect to the other.

In some embodiments, the actuator is the hydraulic actuator including a bladder positioned between the first and second jaws, wherein the bladder is selectively inflatable to move the at least one of the first or second jaws with respect to the other. Alternatively, each of the first and second jaws may be at least partially hollow and selectively inflatable to move the at least one of the first or second jaws with respect to the other.

In some embodiments, the actuator is the screw and nut actuator including a nut attached to an axle, a screw extending through the nut, a first arm pivotably attached to the first jaw and the axle, and a second arm pivotably attached to the second jaw and the axle, wherein rotation of the screw displaces the axle and extends the first and second arms thereby moving the at least one of the first or second jaws with respect to the other.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIGS. 1A to 1B are side views of an exclusion clip system, including an exclusion clip and an exclusion clip delivery and recapture device, according to an exemplary embodiment of the present disclosure;

FIGS. 2A to 2C are side views of a distal end of an exclusion clip delivery and recapture device according to an exemplary embodiment of the present disclosure;

FIGS. 3A to 3B are side views of a distal end of an exclusion clip delivery and recapture device according to an exemplary embodiment of the present disclosure;

FIGS. 4A to 4C are side views of an exclusion clip system, including an exclusion clip and an exclusion clip delivery and recapture device, according to an exemplary embodiment of the present disclosure;

FIGS. 5A to 5E are side views of a distal end of an exclusion clip delivery and recapture device according to an exemplary embodiment of the present disclosure;

FIG. 6 is a fragmented, cross-sectional view of an end effector and an exclusion clip according to an exemplary embodiment of the present disclosure;

FIG. 7 is a fragmented, cross-sectional view of an end effector and an exclusion clip according to an exemplary embodiment of the present disclosure;

FIGS. 8A to 8C are fragmented, cross-sectional views of an end effector and an exclusion clip according to an exemplary embodiment of the present disclosure;

FIGS. 9A to 9C are fragmented, cross-sectional views of an end effector and an exclusion clip according to an exemplary embodiment of the present disclosure;

FIGS. 10A to 10C are fragmented, cross-sectional views of an end effector and an exclusion clip according to an exemplary embodiment of the present disclosure;

FIGS. 11A to 11C are fragmented, cross-sectional views of an end effector and an exclusion clip according to an exemplary embodiment of the present disclosure;

FIGS. 12A to 12C are fragmented, cross-sectional views of an end effector and an exclusion clip according to an exemplary embodiment of the present disclosure;

FIGS. 13A to 13B are fragmented, cross-sectional views of an end effector according to an exemplary embodiment of the present disclosure;

FIGS. 14A to 14B are fragmented, cross-sectional views of an end effector according to an exemplary embodiment of the present disclosure;

FIGS. 15A to 15B are fragmented, cross-sectional views of an end effector according to an exemplary embodiment of the present disclosure;

FIGS. 16A to 16B are fragmented, cross-sectional views of an end effector according to an exemplary embodiment of the present disclosure;

FIGS. 17A to 17B are fragmented, cross-sectional views of an end effector according to an exemplary embodiment of the present disclosure;

FIG. 18 is a fragmented, cross-sectional view of an end effector and an exclusion clip according to an exemplary embodiment of the present disclosure;

FIG. 19A is perspective view of an end effector according to an exemplary embodiment of the present disclosure in a closed configuration;

FIG. 19B is a fragmented, cross-sectional view of the end effector shown in FIG. 19A;

FIG. 19C is a fragmented, cross-sectional view of the end effector shown in FIG. 19A in an open configuration;

FIG. 20A is top perspective view of an end effector according to an exemplary embodiment of the present disclosure in an open configuration;

FIG. 20B is a fragmented, cross-sectional view of the end effector shown in FIG. 20A in an open configuration;

FIG. 21A is perspective view of an end effector according to an exemplary embodiment of the present disclosure in an open configuration;

FIG. 21B is a perspective view of the end effector shown in FIG. 21A in a closed configuration;

FIG. 22A is perspective view of an end effector according to an exemplary embodiment of the present disclosure in a closed configuration;

FIG. 22B is a perspective view of the end effector shown in FIG. 22A in an open configuration;

FIG. 22C is a side view of the end effector shown in FIG. 22A in a closed configuration;

FIG. 22D is a side view of the end effector shown in FIG. 22A in an open configuration;

FIG. 22E is a fragmented, cross-sectional view of the end effector shown in FIG. 22A in a closed configuration;

FIG. 22F is a fragmented, cross-sectional view of the end effector shown in FIG. 22A in an open configuration;

FIG. 23A is perspective view of an end effector according to an exemplary embodiment of the present disclosure in a closed configuration; and

FIG. 23B is a perspective view of the end effector shown in FIG. 23A in an open configuration.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions, or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

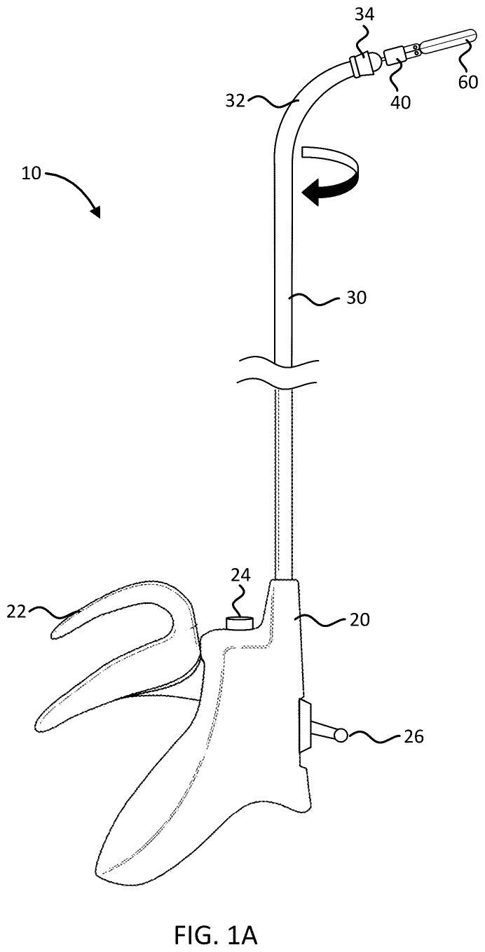

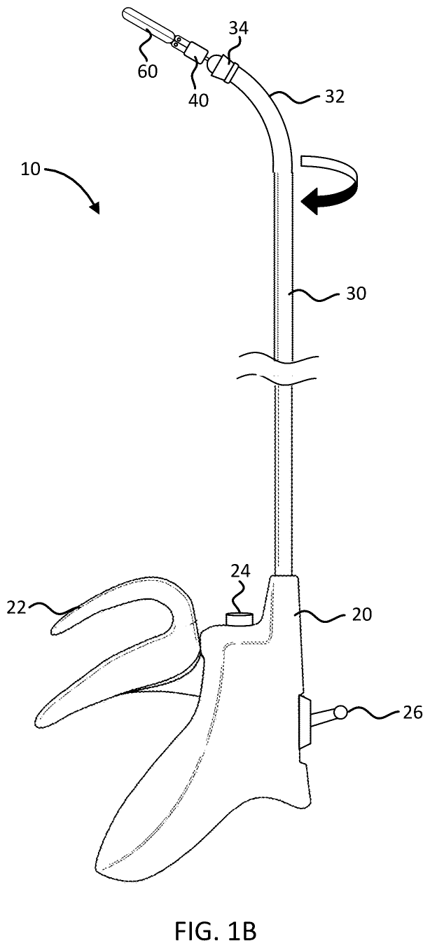

FIGS. 1A to 1B illustrates an exclusion clip delivery and recapture system according to an exemplary embodiment of the present disclosure. The system includes a delivery and recapture device 10 and an exclusion clip 60 releasably retained and controlled by an end effector 40 on a distal end of a shaft 30. The exclusion clip 60 may, for example, be of the type disclosed in U.S. Pat. No. 10,925,615 including biasing members and other features described therein. The device 10 further includes a handle 20 with a jaw controller 22, operable to control jaws of the end effector 40, and a release trigger 24 for releasing the exclusion clip 60 from the device 10. As one skilled in the art will understand, the exclusion clip 60 opens and closes in response to the opening and closing of jaws of the end effector 40.

In the exemplary embodiment, the device 10 has a shaft 30 with a bendable portion 32 which may be selectively bent and/or pivot in response to inputs from the user. For example, the device 10 may have a joystick 26 on the handle 20 for controlling the shaft 30. In the exemplary embodiment, the device 10 further includes a mechanism 34 at a distal end of the shaft 30 for connecting the end effector 40 to the shaft 30. In the exemplary embodiment, the mechanism 34 releasably connects the end effector 40 to the shaft 30 in response to a user input. However, in other embodiments, the end effector 40 is permanently or semi-permanently retained in the mechanism 34.

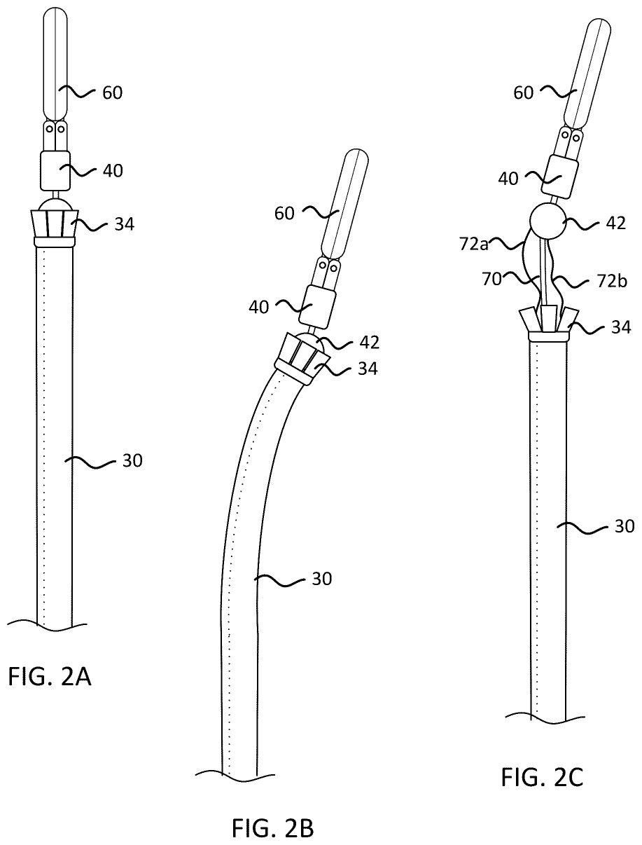

FIGS. 2A to 2C show a distal end of the device 10 including the mechanism 34, the end effector 40, and the exclusion clip 60. The exclusion clip 60 is releasable from the end effector 40, e.g., by actuating the release trigger 24 (see FIGS. 1A-1B). The exclusion clip 60 is typically released after placement on the LAA and may be recaptured if necessary to reposition the exclusion clip 60. However, in some circumstances, it may be advantageous to release the entire end effector 40 after positioning of the exclusion clip 60, e.g., when the position of orientation of the exclusion clip 60 may make it particularly difficult to recapture.

In an exemplary embodiment, the end effector 40 has a proximal end 42 shaped to be received and releasably grasped by the mechanism 34. For example, the proximal end 42 may be ball-shaped, though the shape is not limited thereto. The mechanism 34 may include a plurality of jaws or teeth actuated in response to a user input on the handle 20 (see FIGS. 1A-1B) to open and close around the proximal end 42 of the end effector 40. The mechanism 34 may be selectively actuated to lock the position of the end effector 40 (see FIG. 2A), allow the end effector 40 to rotate (see FIG. 2B), and/or release the end effector 40 (see FIG. 2C).

As shown in FIG. 2C, the end effector 40 may remain loosely connected to the shaft 30 by a control cable 70 for actuating the jaws of the exclusion clip 60, one or more release cords 72a, 72b for releasing the exclusion clip 60, or a combination thereof. However, in some embodiments, the end effector 40 may completely disconnect from and reconnect to the shaft 30.

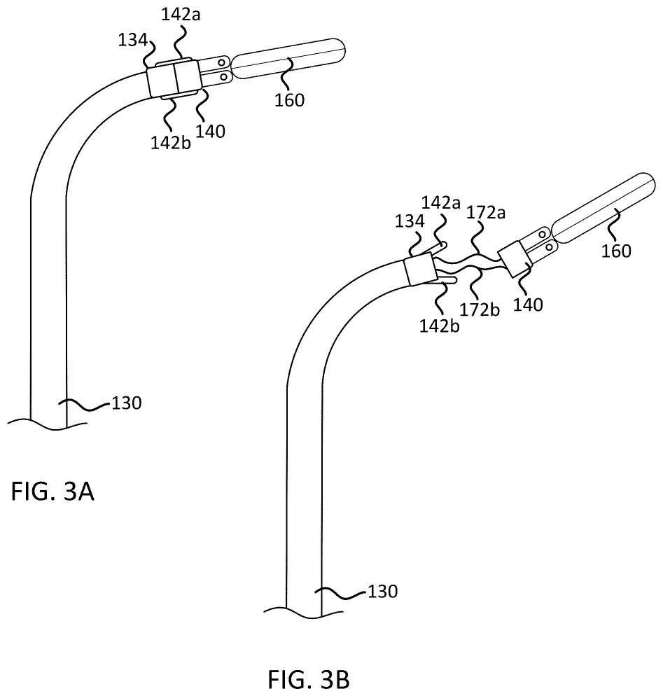

FIGS. 3A to 3B show another exemplary embodiment a distal end of a delivery and recapture device including a mechanism 134 on the end of a shaft 130 (e.g., a bendable shaft), an end effector 140, and an exclusion clip 160. The mechanism 134 has two or more opposing jaws 142a, 142b which selectively grasp and release the end effector 140. The end effector 140 mates against, or at least partially within, an end of the mechanism 134.

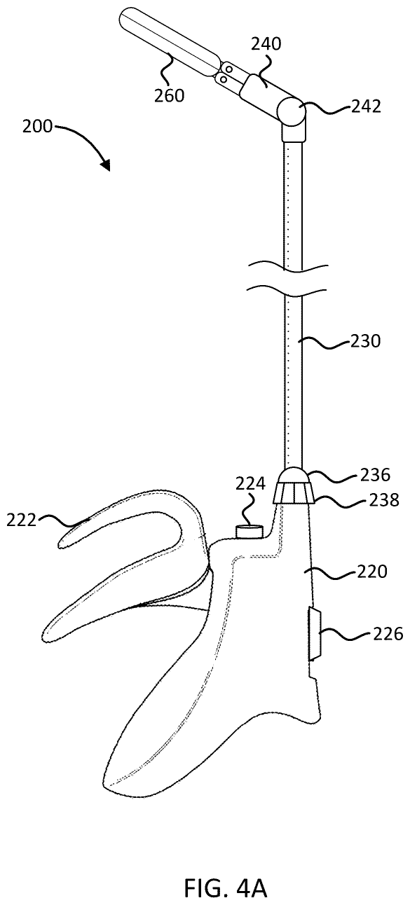

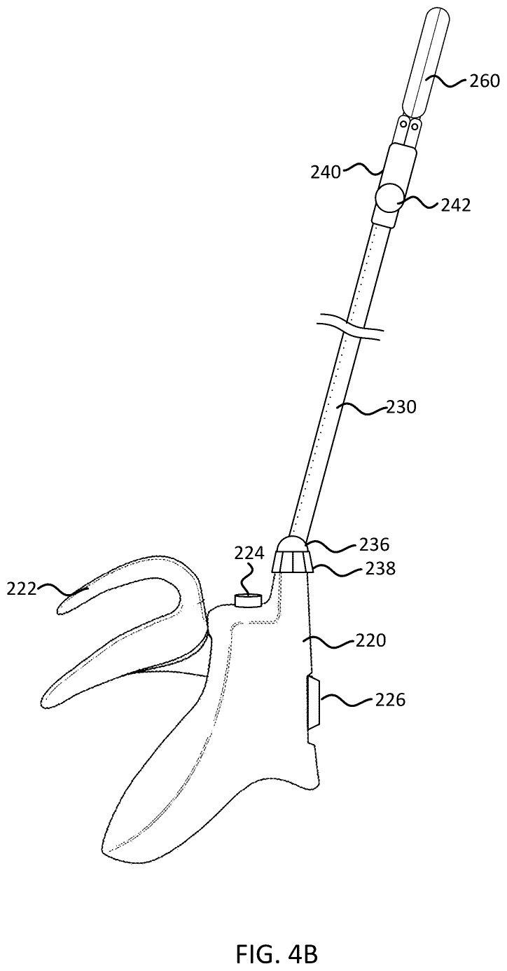

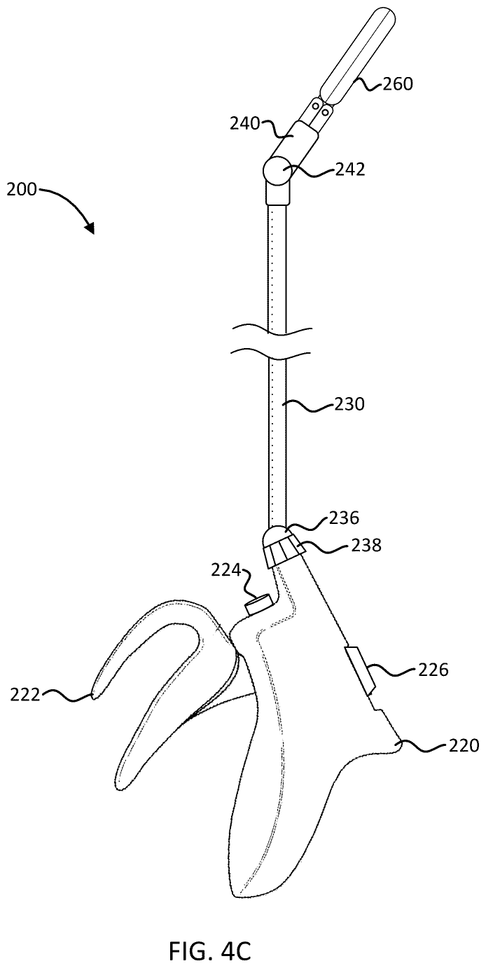

FIGS. 4A to 4C show another exclusion clip delivery and recapture system according to an exemplary embodiment of the present disclosure. The system includes a delivery and recapture device 200 and an exclusion clip 260 releasably retained and controlled by an end effector 240 on a distal end of a shaft 230. The device 200 includes a handle 220 with a jaw controller 222 operable to control jaws of the end effector 240 and a release trigger 224 for releasing the exclusion clip 260 from the device 200.

In the exemplary embodiment, the shaft 230 is moveable and/or pivotable with respect to the handle 220 about a pivot 236. The end effector 240 is also moveable and/or pivotable with respect to the shaft 230 about a pivot 242. In some embodiments, the shaft 230 may be moved with respect to the handle by releasing a lock 238 and manually moving the shaft 230, e.g., with a user's hand or by pressing the shaft 230 against an adjacent surface or object. The end effector 240 may similarly be moved by actuating a lock 226 and manually moving the end effector 240 and/or pressing the end effector 240 against an adjacent surface or object.

In some embodiments, movement of the end effector 240 may be controlled by the relative position of the handle 220 with respect to the shaft 230. For example, as shown in FIG. 4C, tilting the handle 220 downward with respect to the shaft 230 may cause the end effector 240 to tilt upward. Likewise, side to side movements of the handle 220 with respect to shaft 230 may result in left and right movement of the end effector 240.

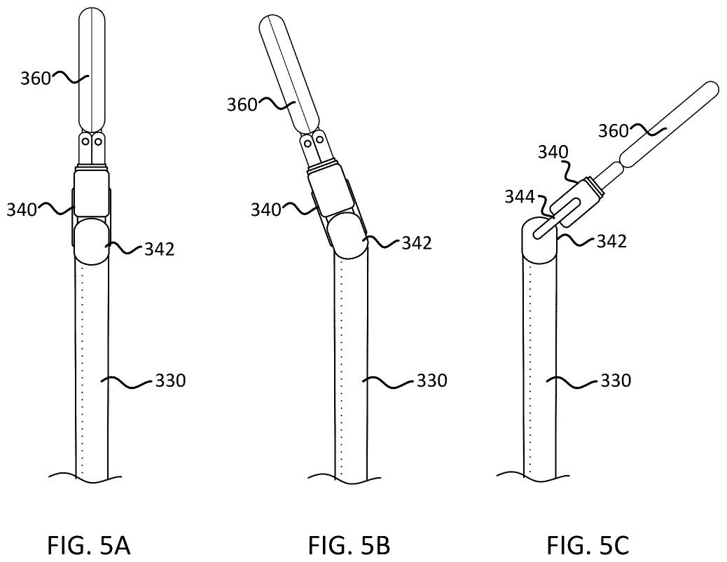

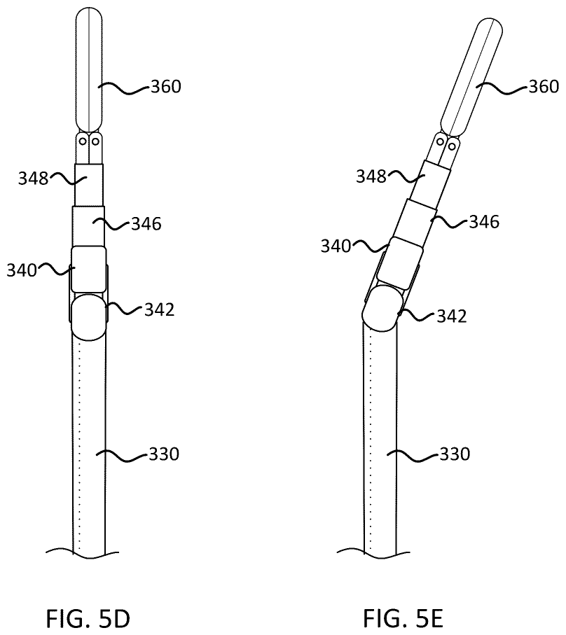

FIGS. 5A to 5E show another exemplary embodiment a distal end of a delivery and recapture device including a shaft 330, an end effector 340, and an exclusion clip 360. In the exemplary embodiment, the end effector 340 has a proximal portion 342 which may be fixed to or pivotable with respect to a distal end of the shaft 330. The proximal portion 342 is also, or alternatively, hingedly attached to the end effector 340 via struts 344. The end effector 340 may be moved with respect to the shaft by passive movement (e.g., pressing the end effector 240 against an adjacent surface or object) or by a control mechanism on a handle.

The end effector 340 is telescoping to allow for the selective extension of the exclusion clip 360 with respect to the distal end of the shaft 330. In the exemplary embodiment, the end effector 340 includes a plurality of nested components 346,348 which may be extended and retracted. In some embodiments, the telescoping elements of the end effector 340 are biased in an extended position by a spring and retained in a collapsed or intermediate position by a lock actuated by a control mechanism on the handle. In some embodiments, the telescoping elements are actuated electrically or hydraulically.

Telescoping is advantageous for placing and recapturing of the exclusion clip 360. For example, the exclusion clip 360 may be released from the end effector 340 (e.g., with or without a tether) while the end effector 340 is at least partially telescoped. The end effector 340 may then be linearly retracted from the exclusion clip 360 using the telescoping feature. The telescoping feature allows for alignment of the end effector 340 and the exclusion clip 360 to be at least partially maintained. If recapture is necessary or desired, the end effector 340 may be linearly telescoped back into engagement the exclusion clip 360.

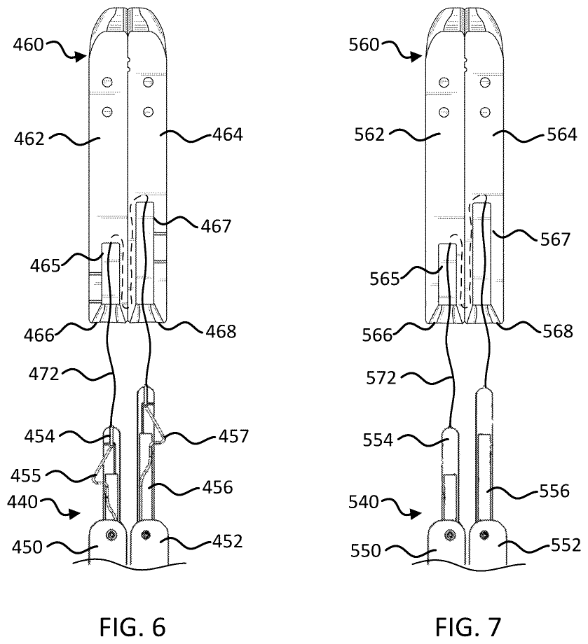

FIG. 6 shows an end effector 440 and an exclusion clip 460 according to an exemplary embodiment of the present disclosure. The end effector 440 includes a first jaw 450 having a first connector 454 and a second jaw 452 having a second connector 456. The exclusion clip 460 includes a first strut 462 having a first connector interface 465 and a second strut 464 having a second connector interface 467 for engaging with the first and second connectors 454,456, respectively. In the exemplary embodiment, the first connector interface 465 on the first strut 462 defines cavity having a proximal end with an opening 466 for receiving the first connector 454. The second connector interface 467 on the second strut 464 defines cavity having a proximal end with an opening 468 for receiving the first connector 454.

In the embodiment shown in FIG. 6, the first and second connectors 454,456 each have a lock 455,457 which may be actuated by a release trigger on the handle (e.g., release trigger 24 shown in FIGS. 1A-1B) to lock and release the exclusion clip 460 to/from end effector 440. The locks 455,457 are in the form of wires which engage into corresponding openings or indentations in the cavities of the first and second connector interfaces 465,467. When the release trigger is actuated, a force is exerted on the wires of the locks 455,457 causing them to straighten or retract and therefore disengage from the exclusion clip 460. However, other means to engage and disengage with the exclusion clip 460 may be used. For example, the lock may also be in the form of a linkage, pivoting pawl, or any other mechanism that retracts when pulled by the wire.

The device further includes a guide mechanism for guiding at least one of the first or second connectors 454,456 into engagement with the exclusion clip 460. In particular, the end effector 440 includes a tether 472 loosely connecting the end effector 440 and the exclusion clip 460. In the exemplary embodiment, the tether 472 is a continuous wire or cord extending from the first connector 454, through the exclusion clip 460, and to the second connector 456. The tether 472 is retractable and extendable from a distal end of at least one of the first or second connectors 454,456.

At least one end of the tether 472 extends through the first or second connector 454,456 and through the end effector 440. The tether 472 may further extend through the shaft and to the handle where it can be selectively controlled by the user. In one embodiment, one end of the tether 472 is fixed to a distal end of one of the first or second connectors 454,456. In another embodiment, the tether 472 is retractable into and extendable from each of the first and second connectors 454,456.

FIG. 7 shows an end effector 540 and an exclusion clip 560 according to another exemplary embodiment of the present disclosure. The end effector 540 includes a first jaw 550 having a first connector 554 and a second jaw 552 having a second connector 556. The exclusion clip 560 includes a first strut 562 and a second strut 564, each having a connector interface 565,567 for engaging with the first and second connectors 554,556. The end effector 540 further includes a tether 572 for retaining a connection to the exclusion clip 560 after it is released.

The embodiment shown in FIG. 7 differs from the embodiment in FIG. 6 in that there is no separate locks to retain and release the exclusion clip 560 from end effector 540. As discussed in more detail below, the exclusion clip 560 is retained by the tether 572. In particular, the exclusion clip 560 may be secured to the end effector 540 by putting tension on the tether 572. At least partially releasing tension on the tether 572 allows the exclusion clip 560 to be released and/or the end effector 540 to be backed away from the exclusion clip 560, e.g., after it is placed in the body. If recapture is necessary, the end effector 540 is guided back into alignment and engagement with the exclusion clip 560 using the tether 572.

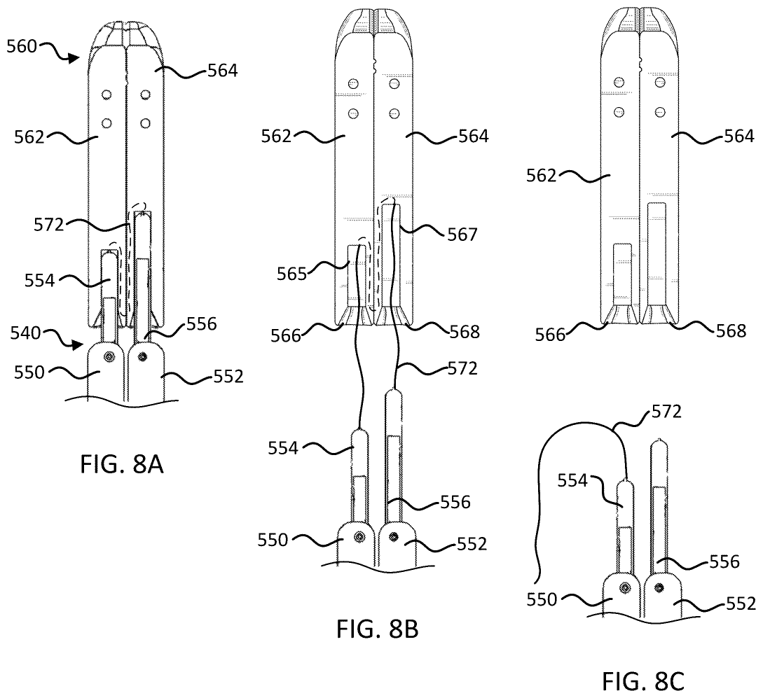

FIGS. 8A to 8C further illustrate the end effector 540 and the exclusion clip 560. FIG. 8A shows the first and second connectors 554,556 engaged in the exclusion clip 560. In the exemplary embodiment, the first and second connectors 554,556 are retained by tension of the tether 572. As shown in FIG. 8B, the exclusion clip 560 may be released from the end effector 540, e.g., after placement and observed while being retained by the tether 572. The release of the exclusion clip 560, and the distance between the end effector 540 and the exclusion clip 560, may be controlled by selectively tensioning and/or releasing the tether 572. If recapture of the exclusion clip 560 is necessary or desired, the end effector 540 may be guided forward along the tether 572.

As shown in FIG. 8C, after proper placement of the exclusion clip 560 is confirmed, the exclusion clip 560 may be fully released from the end effector 540 by releasing or severing the tether 572. In some embodiments, one end of the tether 572 may be pulled through the device until the tether 572 is released. Alternatively, the first and second connectors 554,556 may be reengaged into the exclusion clip 560 and the tether 572 disconnected or severed by imparting sufficient tension on one or both ends. By first reengaging the first and second connectors 554,556 as shown in FIG. 8A, forces on the exclusion clip 560 are negated or at least minimized when tension is applied to the tether 572 because relative movement between the end effector 540 and exclusion clip 560 is avoided. In other embodiments, the delivery and recapture device may include a mechanism to selectively sever the tether 572 (e.g., within the end effector and/or shaft). In some embodiments, the exclusion clip 560 may be held by another device, such as a grasper, while the tether 572 is being tensioned to avoid any movement of the exclusion clip 560. In another embodiment, the tether 572 and/or other tethers described herein may be disconnected by the application of an electric charge that melts a portion of the tether 572 (e.g., within the end effector and/or shaft).

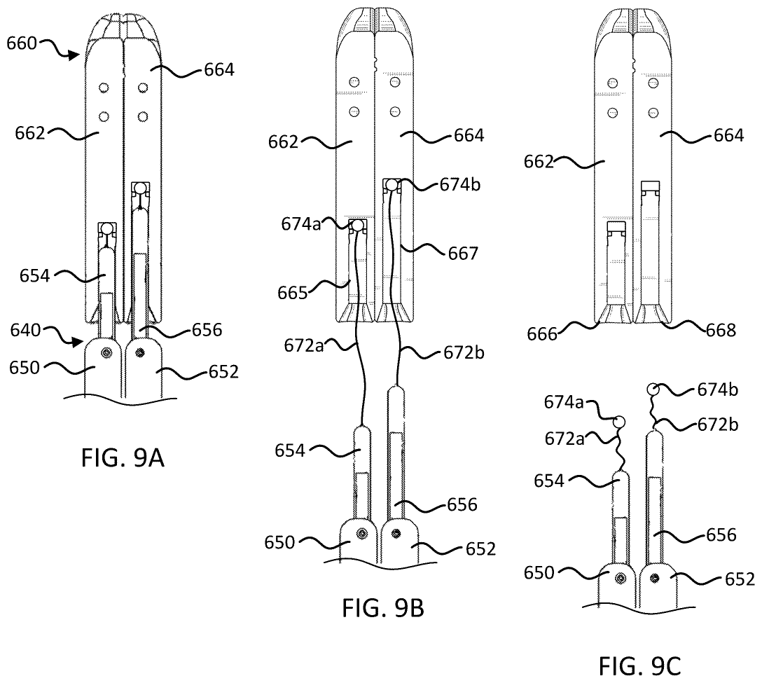

FIGS. 9A to 9C show an end effector 640 and an exclusion clip 660 according to another exemplary embodiment of the present disclosure. Like the previously described embodiment, the end effector 640 includes a first jaw 650 having a first connector 654 and a second jaw 652 having a second connector 656. The exclusion clip 660 includes a first strut 662 and a second strut 664, each having a connector interface 665,667 for engaging with the first and second connectors 654,656.

In this embodiment, one or both of the first and second connectors 654,656 has a separate tether 672a,672b. Each tether 672a,672b has lock configured to engage a retention feature in the cavities of the connector interfaces 665,667. For example, each tether 672a,672b may have a lock in the form of an enlarged distal end 674a,674b (e.g., have a ball-shape) made of a material that is selectively retained by detents, a collar, and/or a change in diameter in a distal end of the respective connector interface 665,667 and can be retained by either a retention feature or by friction between the compressible material and interior surfaces of the cavities of the connector interfaces 665,667. In some embodiments, the material of the enlarged distal end 674a,674b is a compressible material. In other embodiments, the material is an incompressible and/or rigid material. The enlarged distal end 674a,674b may alternatively be a wire form.

FIG. 9A shows the first and second connectors 654,656 engaged in the exclusion clip 660 by tension of the tethers 672a,672b. As shown in FIG. 9B, the exclusion clip 660 may be partially released from the end effector 640, e.g., after placement and observed while being retained by the tether 672a,672b, while a connection is maintained via the tethers 672a,672b. The release of the exclusion clip 660, and the distance between the end effector 640 and the exclusion clip 660 may be controlled by selectively tensioning and/or releasing the tethers 672a,672b. If recapture of the exclusion clip 660 is necessary or desired, the end effector 640 may be guided forward along the tethers 672a,672b.

As shown in FIG. 9C, after proper placement of the exclusion clip 660 is confirmed, the exclusion clip 660 may be fully released from the end effector 640 by releasing the tether 672a,672b. To fully release the exclusion clip 660, the first and second connectors 654,656 may be reengaged into the exclusion clip 660 and the tether 672a,672b tensioned to disengage the enlarged ends 674a,674b from the respective retention features. Having a ball-shaped or other smooth surfaced feature on the ends of the tethers 672a,672b is advantageous for the patient's safety when removing the tethers 672a,672b from the body. In some embodiments, the balled-shaped feature can also be lightly pressed into a cavity in the exclusion clip 660 for attachment of the exclusion clip 660.

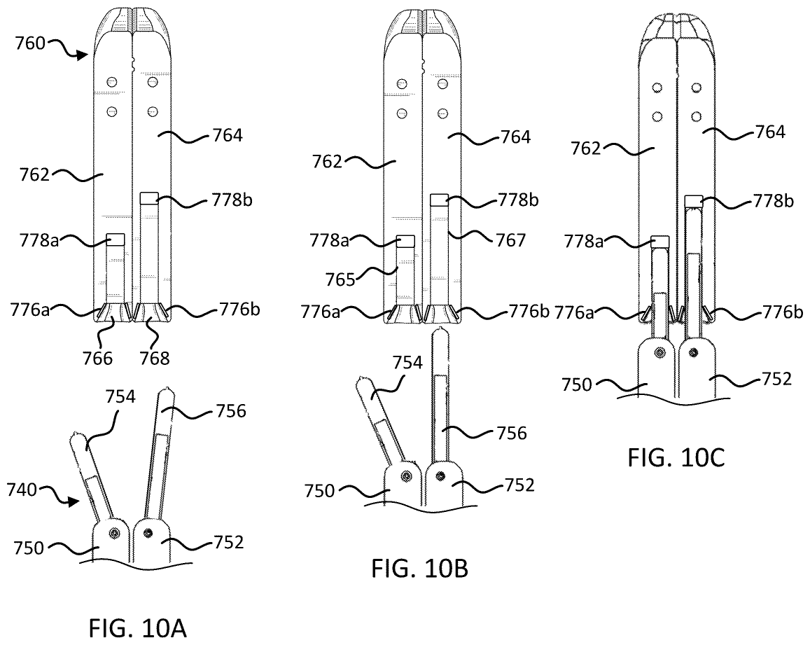

FIGS. 10A to 10C shows an end effector 740 and an exclusion clip 760 according to another exemplary embodiment of the present disclosure. The end effector 740 includes a first jaw 750 having a first connector 754 and a second jaw 752 having a second connector 756. In the exemplary embodiment, the first and second connectors 754,756 are pivotable with respect to the first and second jaws 750,752, respectively. The first and second connectors 754,756 may pivot in one direction (e.g., side to side) or may be free to pivot and/or rotate in any direction. However, pivoting may be constrained within angular range, e.g., such as less than forty-five degrees or less than thirty degrees.

The exclusion clip 760 includes a first strut 762 and a second strut 764, each having a connector interface 765,767 for engaging with the first and second connectors 754,756. In the exemplary embodiment, the first and second connectors 754,756 are guided to and/or retained in the connector interfaces 765,767 using magnets. In some embodiments, the connector interfaces 765,767 have one or more magnets 776a,776b at the openings 766,768 to guide the first and second connectors 754,756 into position. In some embodiments, magnets 778a, 778b are placed at the distal ends of the cavities of the connector interfaces 765,767 to retain the first and second connectors 754,756. In some embodiments, both sets of magnets are provided.

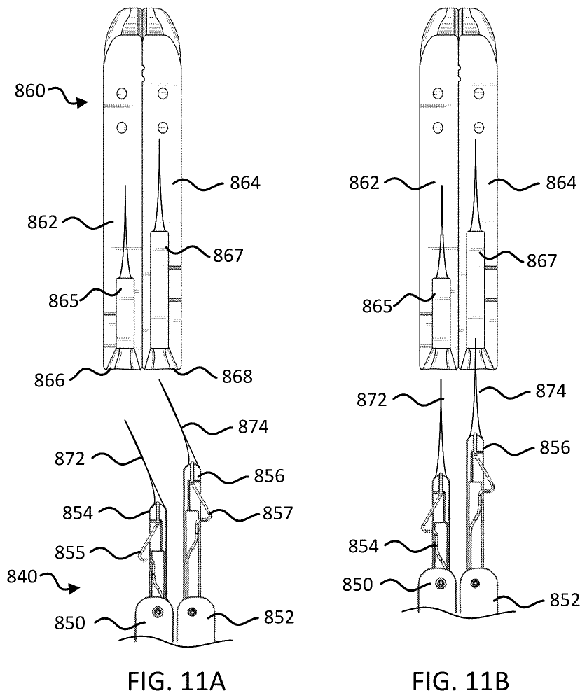

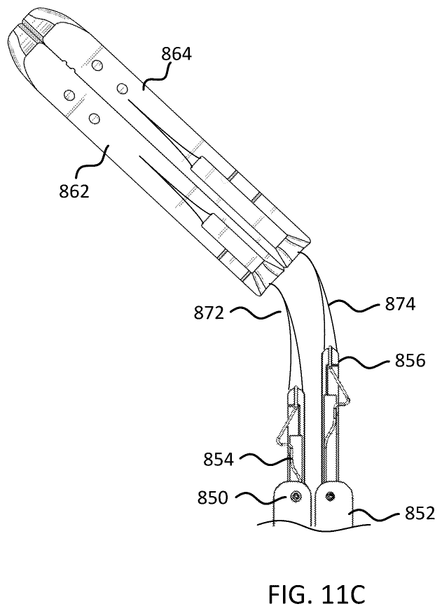

FIGS. 11A to 11C shows an end effector 840 and an exclusion clip 860 according to another exemplary embodiment of the present disclosure. The end effector 840 includes a first jaw 850 having a first connector 854 and a second jaw 852 having a second connector 856. The exclusion clip 860 includes a first strut 862 and a second strut 864, each having a connector interface 865,867 for engaging with the first and second connectors 854,856.

At least one of the first and second connectors 854,856 may have a lock 855,857 to selectively lock the exclusion clip 860 to, and release the exclusion clip 860 from, the end effector 840. The locks 855,857 may be in the form of a wire which engages into openings or indentations in the cavities of the first and second connector interfaces 865,867. When actuated the release trigger is actuated, a force is exerted on wire of the locks 855,857 causing them to straighten or retract and therefore disengage from the exclusion clip 860. However, other means to lock and release the exclusion clip 860 may be employed.

In the exemplary embodiment, first and second connectors 854,856 have flexible and/or selectively turnable extensions 872,874 to facilitate guiding the first and second connectors 854,856 into connector interfaces 865,867. As shown in FIG. 11A, the extensions 872,874 may be turnable in response to a user input. In some embodiments, tension or force may be applied to the wires of the locks 855,857, or separate wires connected directly to the extensions 872,874, to turn the extensions 872,874.

In other embodiments, the extensions 872,874 are flexible but not selectively turnable and retained in an angled position as shown in FIG. 11A or a straight position as shown in FIG. 11B. In some embodiments, the extensions 872,874 are retained at a fixed angle as shown in FIG. 11A but rotatable about axes of the first and second connectors 854,856 to change direction (e.g., passively or in response to a user input). In other embodiments, the extensions 872,874 are bendable in response to a user input as shown in FIG. 11C.

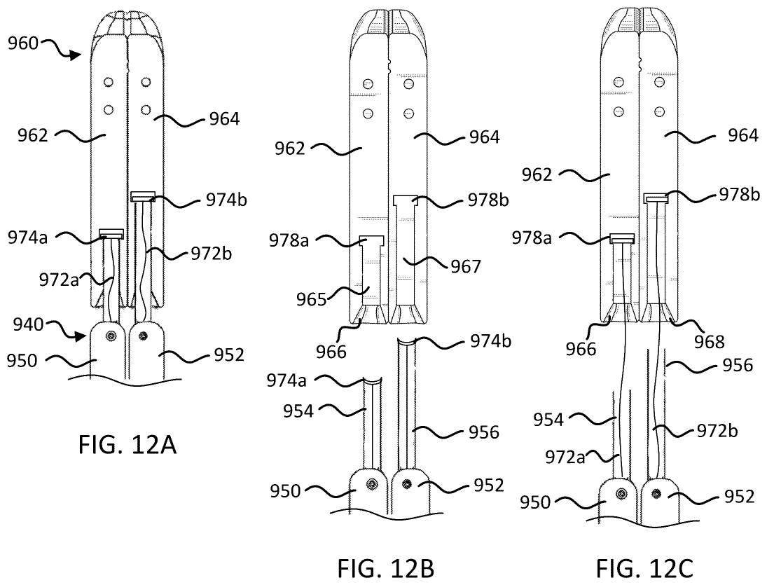

FIGS. 12A to 16B illustrate additional embodiments of locks to releasably secure an exclusion clip to an end effector. FIGS. 12A to 12C show an end effector 940 including a first jaw 950 having a first connector 954 and a second jaw 952 having a second connector 956. At least one of the first and second connectors 954,956 has a lock in the form of a flexible end 974a,974b, having an enlarged diameter, and a tether 972a,972b connected to each respective flexible end 974a,974b.

The exclusion clip 960 includes a first strut 962 and a second strut 964, each having a connector interface 965,967 for engaging with the first and second connectors 954,956. Distal ends of the cavities of the connector interfaces 965,967 include retention features 978a,978b to receive and retain the flexible ends 974a,974b. As shown in FIG. 12A, the flexible ends 974a,974b are retained in the retention features 978a,978b when there is little or no tension on the tethers 972a,972b. Applying tension to the tethers 972a,972b causes the flexible ends 974a,974b to bend inward and disengage from the retention features 978a,978b as shown in FIG. 12B. Releasing the tension causes the flexible ends 974a,974b to straighten to return to their original diameters. Tension may be applied again to reengage and recapture the exclusion clip 960.

In some embodiments, the flexible ends 974a,974b are removably connected to distal ends of the connectors 954,956. As shown in FIG. 12C, tension may be released on the tethers 972a,972b to partially release the exclusion clip 960 while the flexible ends 974a,974b remain engaged in the retention features 978a,978b.

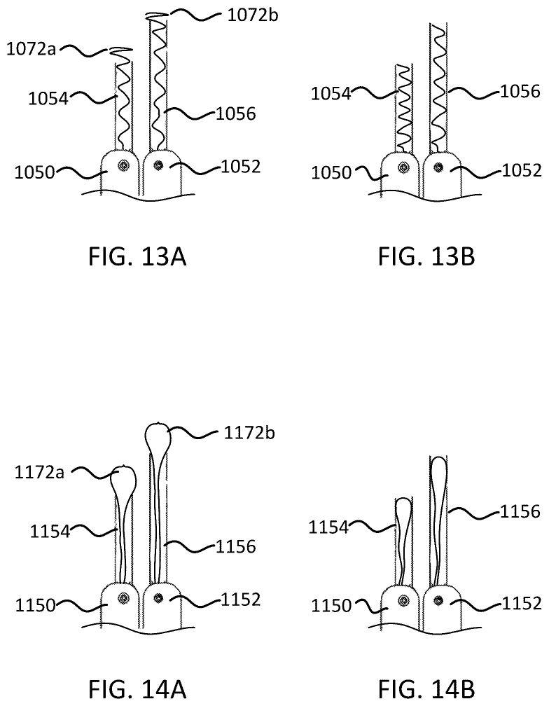

FIGS. 13A to 13B show another embodiment in which at least one of the first and second connectors 1054,1056 includes a lock in the form of a coil 1072a,1072b. A distal end of each coil 1072a,1072b has a diameter greater than a diameter of the connectors 1054,1056 to engage in a retention feature of a respective connector interface. When the coils 1072a,1072b are tensioned by a tether, the distal ends are compressed and pulled into the respective connector 1054,1056 thereby disengaging from the retention feature. The coils 1072a,1072b may also be extended from the ends of the respective connectors 1054,1056 by releasing tension on the tethers to partially release an exclusion clip while the coils 1072a,1072b remain engaged in respective retention features. In some embodiments, at least one or both of the coils 1072a,1072b engages in respective connector interfaces by friction, i.e., without requiring any retention features in cavities of the connector interfaces.

FIGS. 14A to 14B show an embodiment in which at least one of the first and second connectors 1154,1156 includes a lock in the form of a balloon 1172a,1172b. When the balloons 1172a,1172b are outside of the connectors 1054,1056, the balloons 1172a,1172b have a diameter greater than a diameter of the connectors 1054,1056 to engage in a retention feature of a respective connector interface. The balloons 1172a,1172b may be disengaged from the retention features by pulling the balloons 1172a,1172b into the respective connectors 1154,1156. The balloons 1172a,1172b may also be extended from the ends of the respective connectors 1154,1156 by releasing tension on the tethers to partially release an exclusion clip while the balloons 1172a, 1172b remain engaged in respective retention features. In some embodiments, the balloons 1172a, 1172b may be at least partially inflated with a liquid or gas. In some embodiments, the balloon 1172a, 1172b is at least partially comprised of or filled with an elastomeric material without requiring inflation. In some embodiments, at least one or both of the balloons 1172a, 1172b engages in respective connector interfaces by friction, i.e., without requiring any retention features in cavities of the connector interfaces.

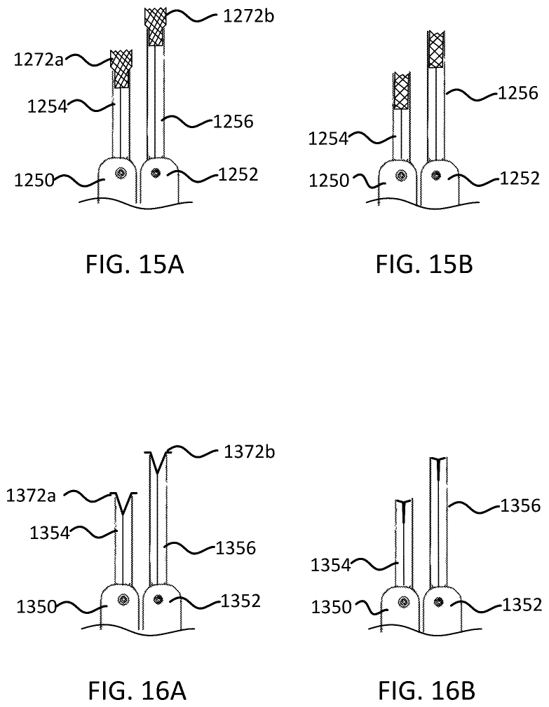

FIGS. 15A to 15B show an embodiment in which at least one of the first and second connectors 1254,1256 includes a lock in the form of a collapsible strut 1272a, 1272b. For example, each strut 1272a,1272b may have a stent-like structure that expands to a diameter greater than a diameter of the connectors 1254,1256 to engage in a retention feature of a respective connector interface. When the struts 1272a,1272b are tensioned by a tether, the distal ends are compressed and pulled into the respective connector 1254,1256 thereby disengaging from the retention feature. The struts 1272a,1272b may also be extended from the ends of the respective connectors 1254,1256 by releasing tension on the tethers to partially release an exclusion clip while the struts 1272a,1272b remain engaged in respective retention features. In some embodiments, at least one or both of the struts 1272a,1272b engages in respective connector interfaces by friction, i.e., without requiring any retention features in cavities of the connector interfaces.

FIGS. 16A to 16B show an embodiment in which at least one of the first and second connectors 1354,1356 includes a lock in the form of a retention clip 1372a,1372b. Each retention clip 1372a,1372b may be comprised of an elastic metal (e.g., nitinol) having a distal end that expands to a diameter greater than a diameter of the connectors 1354,1356 to engage in a retention feature of a respective connector interface. When the retention clips 1372a,1372b are tensioned by a tether, the clips 1372a,1372b are compressed and pulled into the respective connector 1354,1356 thereby disengaging from the retention feature. The clips 1372a,1372b may also be extended from the ends of the respective connectors 1354,1356 by releasing tension on the tethers to partially release an exclusion clip while the struts 1372a,1372b remain engaged in respective retention features. In some embodiments, at least one or both of the retention clips 1372a,1372b engages in respective connector interfaces by friction, i.e., without requiring any retention features in cavities of the connector interfaces.

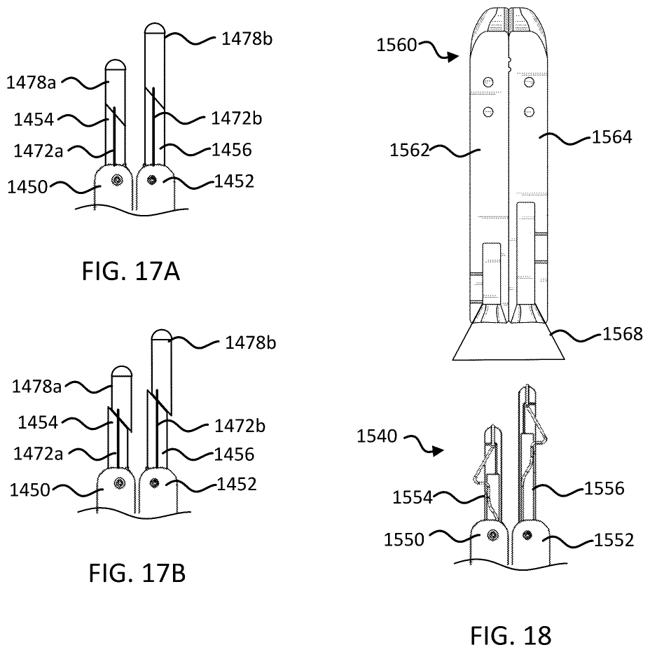

FIGS. 17A to 17B show an embodiment in which at least one of the first and second connectors 1454,1456 includes a lock in the form of a distal end 1478a,1478b that is displaceable in response to a tether 1472a,1472b. In the exemplary embodiment, the distal ends 1478a,1478b of the respective connectors 1454,1456 have angled proximal ends. When the tethers 1472a,1472b are not tensioned or lightly tensioned, the distal ends 1478a,1478b are in line with the rest of the respective connectors 1454,1456 as shown in FIG. 17A. When tension is applied to the tethers 1472a,1472b, the distal ends 1478a,1478b as displaced along the angled proximal surfaces resulting in a misalignment with the rest of the respective connectors 1454,1456 as shown in FIG. 17B. When the connectors 1454,1456 are positioned within an exclusion clip, the misalignment locks the connectors 1454,1456 in the exclusion clip by friction between the misaligned connectors 1454,1456 and interior walls of cavities in the exclusion clip. Alternatively, the misaligned connectors 1454,1456 may engage retention features in the cavities of the exclusion clip. Other variations of two-part connectors 1454,1456 which at least partially displace or expand to secure an exclusion clip may also be employed.

FIG. 18 shows an end effector 1540 and an exclusion clip 1560 according to an exemplary embodiment of the present disclosure. In the exemplary embodiment, the exclusion clip 1560 has a cone 1568 to guide and facilitate engagement of the connectors 1554,1556. In some embodiments, the cone 1568 is a single cone. In other embodiments, each opening on exclusion clip 1560 has a separate cone. The cone 1568 may be pre-attached to the exclusion clip 1560 or attached during a medical procedure if necessary (e.g., with a snap on connector or magnets). The cone 1568 may be removed after the exclusion clip 1560 is in place. Alternatively, the cone 1568 may be comprised of a soft, flexible material, which can remain on the exclusion clip 1560 without impinging on adjacent structures, or a dissolvable material.

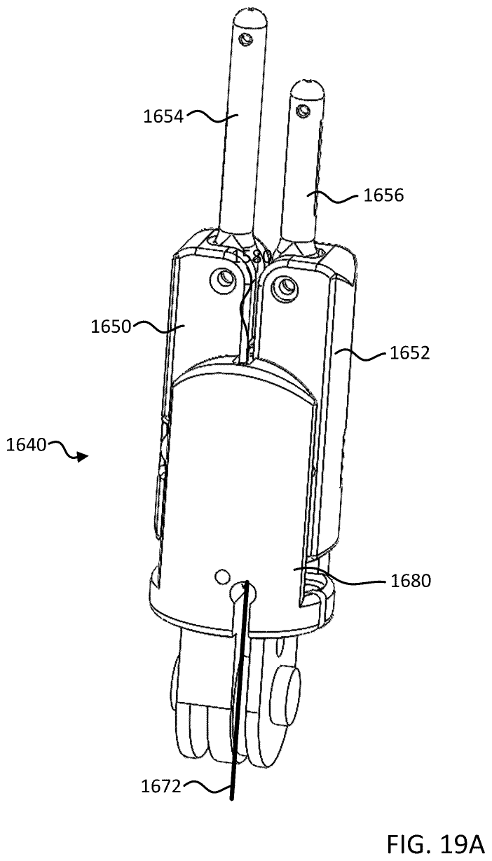

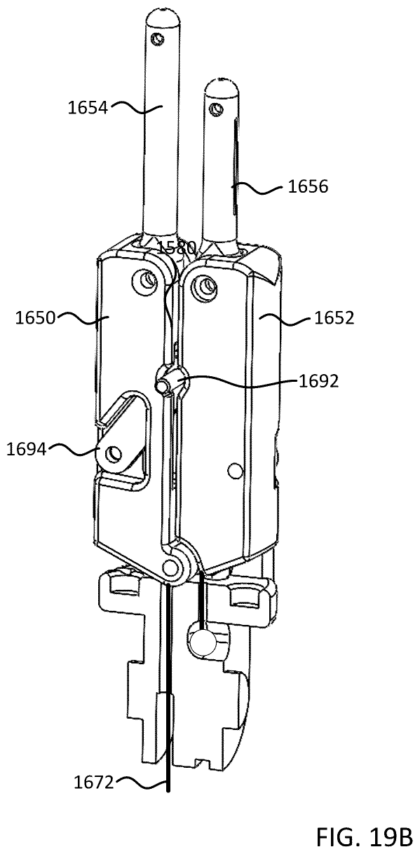

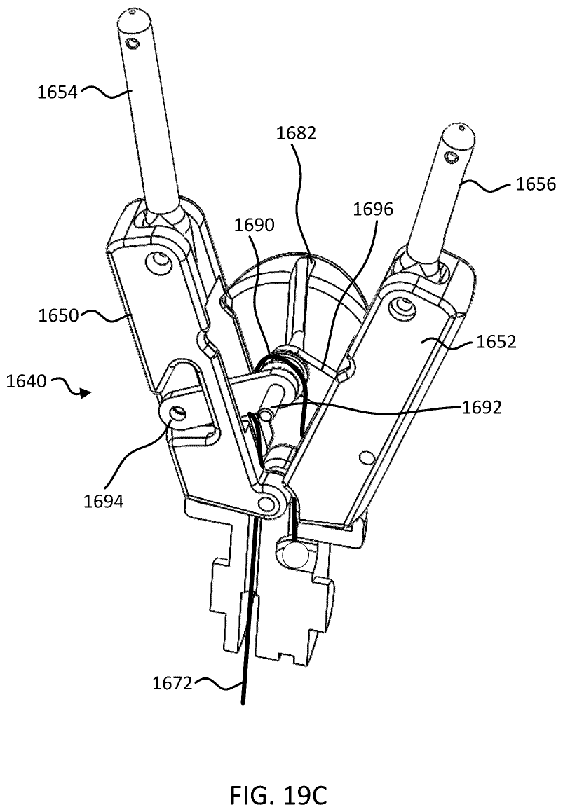

FIGS. 19 to 23B illustrate end effectors with various actuators or actuation mechanisms according to exemplary embodiments of the present disclosure.

FIGS. 19A to 19C illustrate an end effector 1640 according to an exemplary embodiment of the present disclosure. The end effector 1640 includes a first jaw 1650 having a first connector 1654 and a second jaw 1652 having a second connector 1656. The end effector 1640 is actuated by a pull wire 1672 which extends over a pulley 1690. A distal end of the pull wire 1672 may be fixedly retained in the end effector 1640. In the exemplary embodiment, the distal end of the pull wire 1672 has a retention feature which engages in a pocket in the end effector 1640.

As shown in FIG. 19C, the pulley 1690 may rotate around an axle 1692 which is slidably engaged in tracks on the inside surfaces of two opposing plates 1680,1682. A first arm 1694 is pivotably attached to both the first jaw 1650 and the axle 1692. A second arm 1696 is pivotably attached to both the second jaw 1652 and the axle 1692. In some embodiments, the pulley 1690 and the axle 1692 are fixed in rotation such that the pull wire 1672 slides over the pulley 1690. In such embodiments, the pulley 1690 may be any structure having an upper surface configured to receive the pull wire 1672, such as a structure have an at least partially curved upper surface.

The end effector 1640 is biased in a closed position (e.g., with a spring). In response to tension on the pull wire 1672, the axle 1692 slides downward in the tracks causing the first and second arms 1694,1696 to straighten and open the first and second jaws 1650,1652. The end effector 1640 may be closed by releasing tension on the pull wire 1672.

The pull wire 1672 may be actuated manually, e.g., via a mechanical mechanism on the handle (e.g., jaw controller 22). The pull wire 1672 also actuated by an electric motor, or any electro-mechanical system that can transmit motion. An electric motor may be positioned in the handle, in the distal end of the shaft or in the end effector 1640. The motor preferably has a manual override and/or release to control the end effector 1640 in the event of a malfunction with the motor.

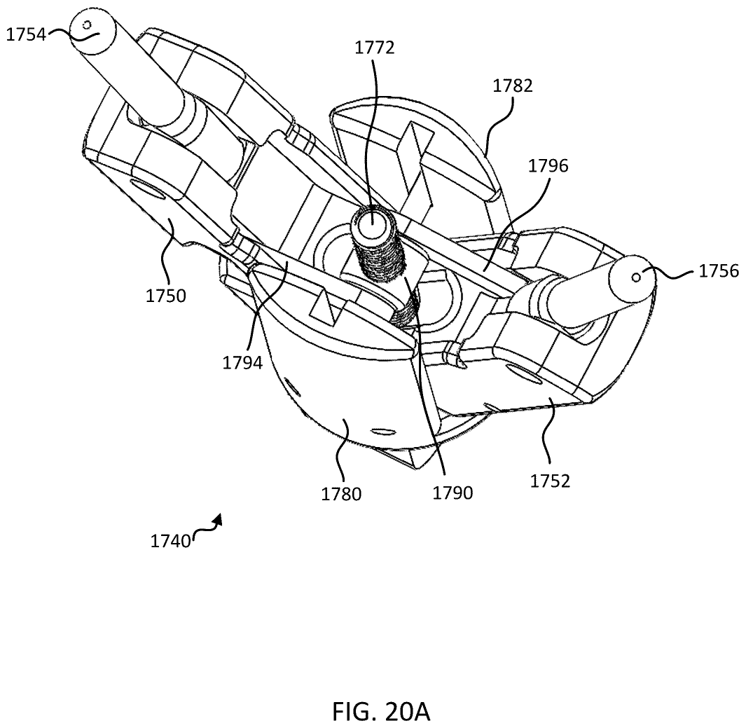

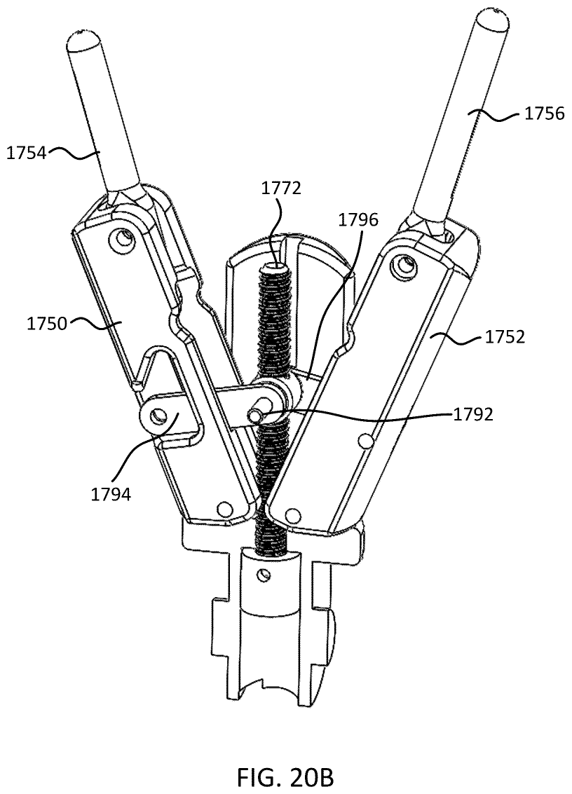

FIGS. 20A and 20B illustrate an end effector 1740 according to an exemplary embodiment of the present disclosure. The end effector 1740 includes a first jaw 1750 having a first connector 1754 and a second jaw 1752 having a second connector 1756. The end effector 1740 is actuated by a screw 1772 which engages with a nut 1790. The nut 1790 is positioned around an axle 1792 which is slidably engaged in tracks on the inside surfaces of two opposing plates 1780,1782. A first arm 1794 is pivotably attached to both the first jaw 1750 and the axle 1792. A second arm 1796 is pivotably attached to both the second jaw 1752 and the axle 1792.

In response to a rotation of the screw 1772, the axle 1792 slides downward in the tracks causing the first and second arms 1794,1796 to straighten and open the first and second jaws 1750,1752. The end effector 1740 may be closed by rotating the screw 1772 in an opposite direction. As with the previous embodiment, the screw 1772 may be actuated manually or with a motor or other mechanical/electro-mechanical system (e.g., a spring-loaded motor). In some embodiments, a continued rotation of the screw 1772 after the end effector 1740 is closed causes the end effector 1740 to release an exclusion clip. In some embodiments, the screw 1772 is configured with multiple pitches or leads to allow the end effector 1740 to change the speed at which the end effector 1740 opens and closes. For example, the speed at which the end effector 1740 may change to a slower speed when the end effector 1740 approaches a fully closed position due to the pitch or lead of the screw 1772 changing along the length of the screw 1772.

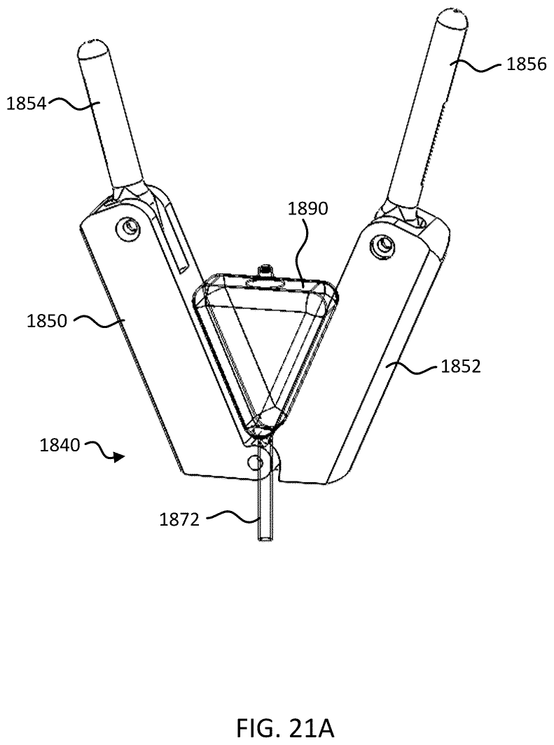

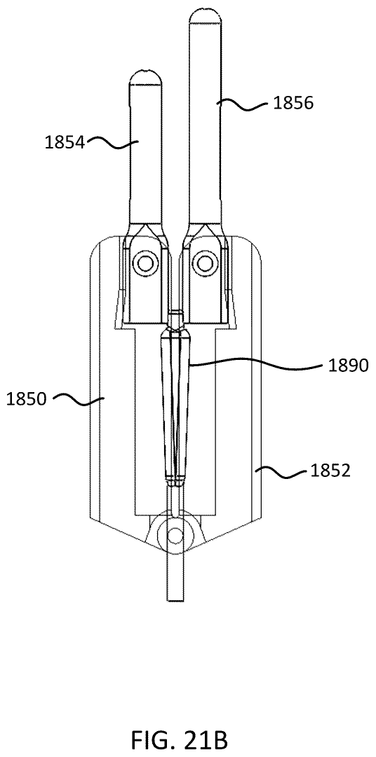

FIGS. 21A to 21B illustrate an end effector 1840 according to an exemplary embodiment of the present disclosure. The end effector 1840 includes a first jaw 1850 having a first connector 1854 and a second jaw 1852 having a second connector 1856. The end effector 1840 is actuated by a hydraulic line 1872 which fills and evacuates a bladder 1890. A gas or fluid may be used, such as air, water or saline. Positive pressure is applied to inflate the bladder 1890 and open the end effector 1840. Negative pressure is applied to evacuate the bladder 1890 and close the end effector 1840.

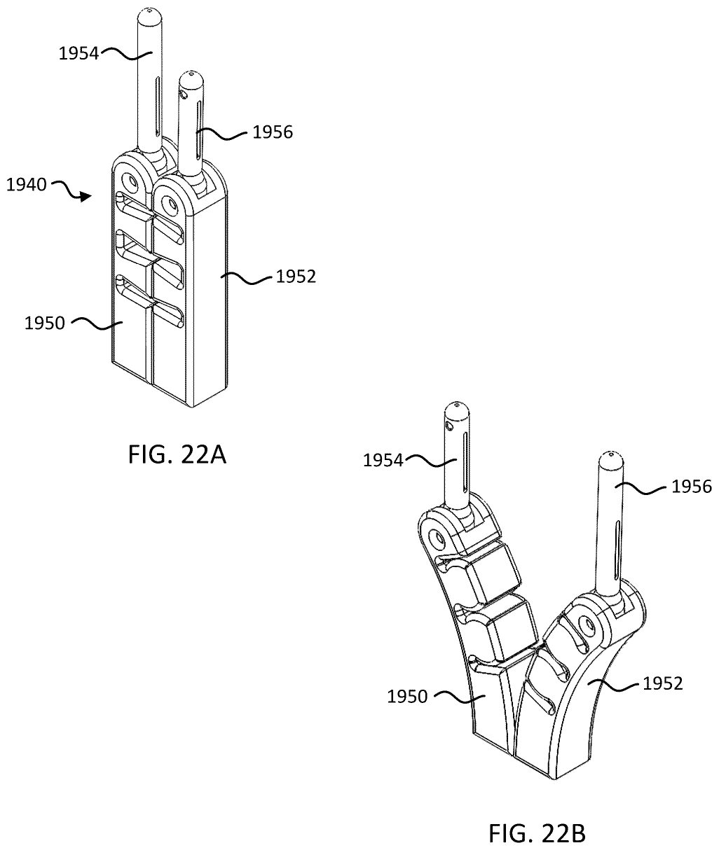

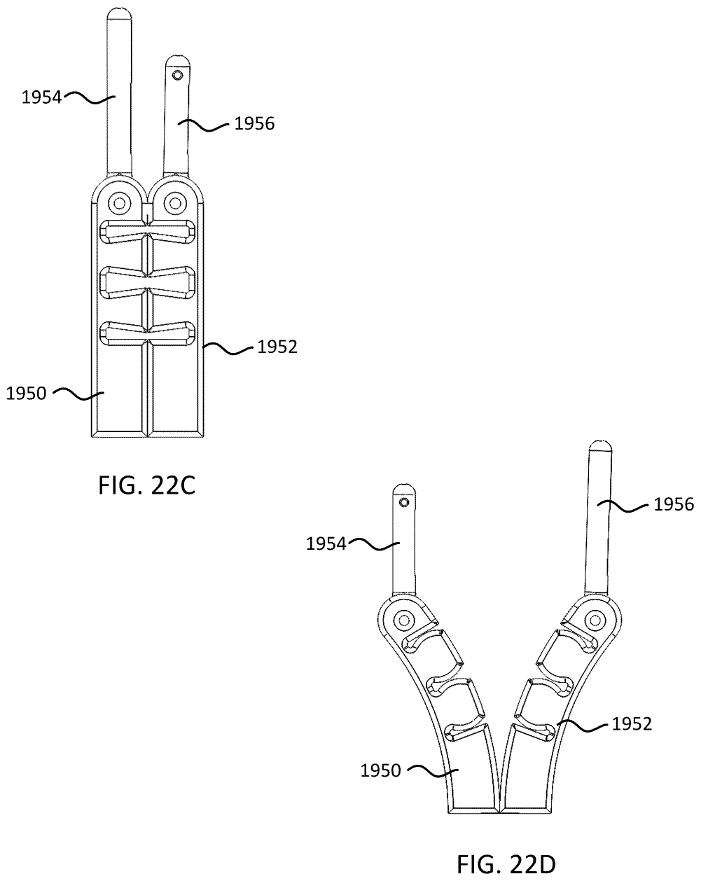

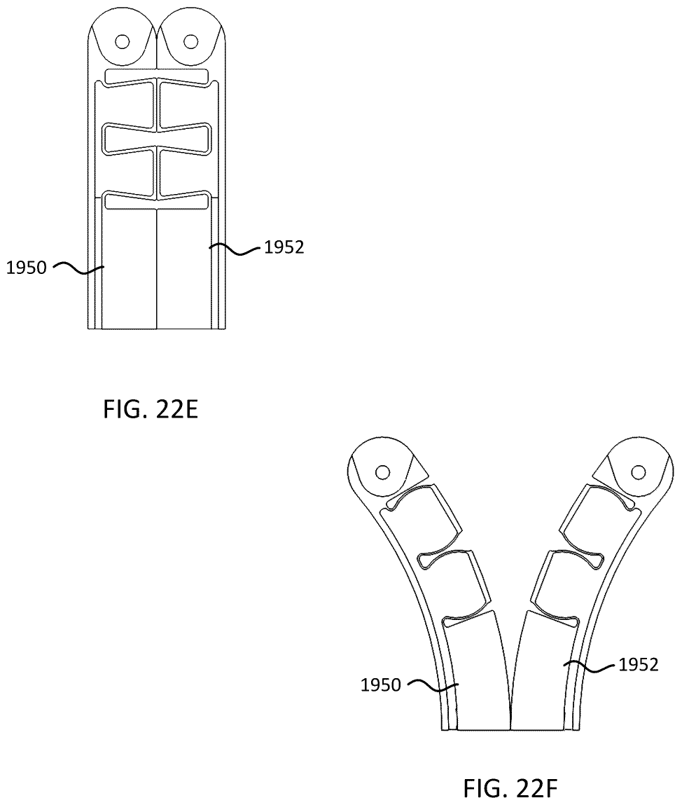

FIGS. 22A to 22F illustrate an end effector 1940 according to an exemplary embodiment of the present disclosure. The end effector 1940 includes a first jaw 1950 having a first connector 1954 and a second jaw 1952 having a second connector 1956. The jaws 1950,1952 are at least partially hollow for receiving a gas or fluid, e.g., via a hydraulic line. The jaws 1950,1952 are comprised of a flexible/elastic material and portions are designed with a reduced wall thickness to expand and contract in response to hydraulic pressure.

Each jaw 1950,1952 has a plurality of inwardly extending bladders positioned adjacent to one another. As shown in FIGS. 22A and 22C, before hydraulic pressure is introduced, the bladders are at least partially spaced apart from one another. As shown in FIGS. 22B and 22D, as hydraulic pressure is introduced to the jaws 1950,1952, adjacent walls of the bladders expand causing the jaws 1950,1952 to open. In one embodiment, the outer surface or spine of each jaw 1950,1952 (opposite the bladders) is comprised of a stiffer material such as carbon fiber, Nitinol, or a harder durometer plastic/polymer and the bladders are comprised of a softer polymer.

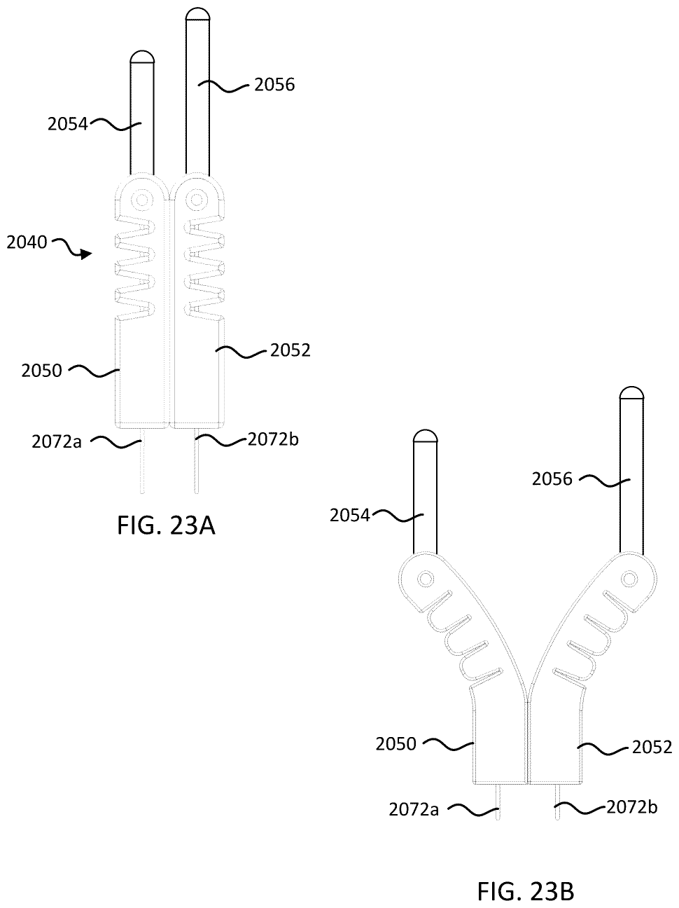

FIGS. 23A and 23B illustrate an end effector 2040 according to an exemplary embodiment of the present disclosure. The end effector 2040 includes a first jaw 2050 having a first connector 2054 and a second jaw 2052 having a second connector 2056. Each jaw 2050,2052 has a plurality of outwardly extending features positioned adjacent to and spaced apart from one another and a pull cord 2072a,2072b. By putting tension on the pull cords 2072a,2072b, the outwardly extending features on jaw 2050,2052 at least partially collapse against one another allowing the jaws 2050,2052 to bend into an open configuration.

As shown throughout the drawings, like reference numerals designate like or corresponding parts. While illustrative embodiments of the present disclosure have been described and illustrated above, it should be understood that these are exemplary of the disclosure and are not to be considered as limiting. Additions, deletions, substitutions, and other modifications can be made without departing from the spirit or scope of the present disclosure. Accordingly, the present disclosure is not to be considered as limited by the foregoing description.

What is claimed is:

1. An exclusion clip delivery and recapture system, comprising:
an exclusion clip including a first strut having a first connector interface and a second strut having a second connector interface; and
a delivery and recapture device for the exclusion clip, comprising:
a handle including a jaw control;
a shaft having a proximal end connected to the handle; and
an end effector, connected to a distal end of the shaft, comprising first and second jaws operatively connected to the jaw control to move at least one of the first or second jaws with respect to the other, a first connector on the first jaw for engagement with the first connector interface of the exclusion clip, and a second connector on the second jaw for engagement with the second connector interface of the exclusion clip; and
a guide mechanism for guiding at least one of the first or second connectors into engagement with the exclusion clip.

2. The system according to claim 1, wherein the guide mechanism includes a tether extending from at least one of the first connector or the second connector.

3. The system according to claim 1, wherein the guide mechanism includes a tether extending from the first connector, through the exclusion clip, to the second connector, and wherein the tether is selectively releasable to disconnect the exclusion clip from the first and second connectors.

4. The system according to claim 1, wherein each of the first and second connector interfaces includes a cavity having a proximal end with an opening for receiving the respective one of the first or second connector and a closed distal end.

5. The system according to claim 4, wherein the guide mechanism includes a first tether extending from the first connector, a distal end of the first tether being removably attached to the closed distal end of the cavity of the first connector.

6. The system according to claim 5, wherein the guide mechanism further includes a second tether extending from the second connector, a distal end of the second tether being removably attached to the closed distal end of the cavity of the second connector.

7. The system according to claim 5, wherein the distal end of the first tether includes a lock configured to engage in a retention feature in the distal end of the cavity.

8. The system according to claim 7, wherein the lock is one of a bendable end piece, a coil, a balloon, a collapsable strut, or a retention clip.

9. The system according to claim 4, wherein at least one of the first and second connectors has a lock in a form of a distal end that is displaceable laterally in response to tension from a tether.

10. The system according to claim 4, wherein the guide mechanism includes at least one cone on a proximal end of the exclusion clip.

11. The system according to claim 4, wherein the guide mechanism includes at least one magnet; and wherein the at least one magnet is positioned at the closed distal end of the cavity of at least one of the first connector interface or the second connector interface, or the at least one magnet is positioned at the proximal end of the cavity of at least one of the first connector interface or the second connector interface.

12. The system according to claim 4, wherein the guide mechanism includes a flexible extension extending from at least one of the first connector or the second connector.

13. The system according to claim 12, wherein the flexible extension is selectively turnable or bendable in response to a user input.

14. The system according to claim 1, wherein the end effector is removably connected to the distal end of the shaft, wherein the distal end of the shaft includes a mechanism to grasp a proximal end of the end effector.

15. The system according to claim 1, wherein the end effector is rotatably connected to the distal end of the shaft, and the end effector is telescoping distal to the rotatable connection.

16. The system according to claim 1, further comprising an actuator for moving the at least one of the first or second jaws with respect to the other in response to the jaw control, the actuator including one of a pulley actuator, a hydraulic actuator, or a screw and nut actuator.

17. The system according to claim 16, wherein the actuator is the pulley actuator, the pulley actuator including pulley on an axle, a wire extending over the pulley, a first arm pivotably attached to the first jaw and the axle, and a second arm pivotably attached to the second jaw and the axle, wherein tensioning the wire over the pulley displaces the axle and extends the first and second arms, thereby moving the at least one of the first or second jaws with respect to the other.

18. The system according to claim 16, wherein the actuator is the hydraulic actuator,
wherein the hydraulic actuator includes a bladder positioned between the first and second jaws, wherein the bladder is selectively inflatable to move the at least one of the first or second jaws with respect to the other, or
wherein each of the first and second jaws is at least partially hollow and selectively inflatable to move the at least one of the first or second jaws with respect to the other.

19. An exclusion clip delivery and recapture system, comprising:
an exclusion clip including a first strut having a first connector interface and a second strut having a second connector interface; and
a delivery and recapture device comprising first and second jaws movable with respect to one another, a first connector on the first jaw for engagement with the first connector interface of the exclusion clip, and a second connector on the second jaw for engagement with the second connector interface of the exclusion clip;
a lock for selectively locking the exclusion clip on, and selectively releasing the exclusion clip from, the delivery and recapture device; and
a guide mechanism for guiding at least one of the first or second connectors into engagement with the delivery and recapture device to recapture the exclusion clip after being selectively released.

20. The system according to claim 19, wherein the guide mechanism includes a tether extending from at least one of the first connector or the second connector, wherein the tether is retractable guiding at least one of the first or second connectors into engagement with the delivery and recapture device.

21. The system according to claim 19, wherein each of the first and second connector interfaces includes a cavity having a proximal end with an opening for receiving the respective one of the first or second connector and a closed distal end, wherein the guide mechanism includes a first tether extending from the first connector, a distal end of the first tether being removably attached to the closed distal end of the cavity of the first connector.

22. The system according to claim 21, wherein the distal end of the first tether is removably attached to the closed distal end of the cavity with the lock.

23. The system according to claim 22, wherein the lock is one of a bendable end piece, a coil, a balloon, a collapsable strut, or a retention clip.
